(19) United States
(12) Patent Application Publication
(10) Pub. No.: US 2019/0300875 A1
(43) Pub. Date: Oct. 3, 2019

(54) BIC INHIBITOR OF CRY-CRY AND CRY-CIB OLIGOMERIZATION/CLUSTERING

(71) Applicant: The Regents of the University of California, Oakland, CA (US)

(72) Inventors: Chentao Lin, Sherman Oaks, CA (US); Yoshito Oka, Fuzhou, Fujian (CN); Qin Wang, Los Angeles, CA (US)

(73) Assignee: The Regents of the University of California, Oakland, CA (US)

(21) Appl. No.: 16/317,019

(22) PCT Filed: Jul. 11, 2017
(86) PCT No.: PCT/US2017/041504

§ 371 Date: Jan. 10, 2019

Related U.S. Application Data
(60) Provisional application No. 62/360,862, filed on Jul. 11, 2016.

(51) Int. Cl. C12N 15/10; C07K 14/47; C12N 5/07; G01N 33/50

(52) U.S. Cl. CPC C12N 15/1079; C07K 14/4702; C07K 2319/70; G01N 33/5005; C12N 5/06

(57) ABSTRACT

The invention includes methods for modulating a reaction dependent on a blue light-dependent protein interaction between a CRY protein and a CRY-signaling protein. The method comprises combining a CRY protein and a CRY-signaling protein with a BIC protein, wherein the BIC protein modulates the blue light-dependent interaction between the CRY protein and the CRY-signaling protein. Embodiments of the invention include compositions of matter comprising a CRY gene or protein, a CRY-signaling gene or protein, and a BIC gene or protein (optionally linked to a heterologous nucleic acid or amino acid sequence), wherein the BIC protein modulates a blue light-dependent interaction between the CRY protein and the CRY-signaling protein.

### CROSS-REFERENCE TO RELATED APPLICATIONS

This application is a U.S. national stage entry of International Application No. PCT/US2017/041504, which claims priority under Section 119(e) from U.S. Provisional Application Ser. No. 62/360,862, filed Jul. 11, 2016, entitled “BIC INHIBITOR OF CRY-CRY AND CRY-CIB OLIGOMERIZATION/CLUSTERING” by Chentao Lin, et al., the contents of which are incorporated herein by reference.

### STATEMENT REGARDING FEDERALLY SPONSORED RESEARCH AND DEVELOPMENT

This invention was made with Government support under R01GM056265, awarded by the National Institutes of Health (NIH). The Government has certain rights in the invention.

### SEQUENCE LISTING

The instant application contains a Sequence Listing which has been submitted electronically in ASCII format and is hereby incorporated by reference in its entirety. Said ASCII copy, created on Aug. 11, 2017, is named 30435_315WOU1_SL.txt and is 16,442 bytes in size.

### TECHNICAL FIELD

The invention relates to optogenetics and in particular, methods and materials useful to modulate and control cryptochrome functions in a wide variety of contexts.

### BACKGROUND OF THE INVENTION

Blue light-dependent cyptochrome protein (CRY)—cryptochrome-interacting basic helix-loop-helix1 protein (CIB) interaction is a state-of-the art technology in optogenetics (see, e.g. Science 2008, 322:1535; Nature Method, 2010, 7:973; PNAS 2012, 109 (35) E2316; Nature Method 2013, 10:249; Nature, 2013500:472; Nature Method, 2014: 11:633; Nature Communications, 2014, 5:4925; and Science, 2014, 345:313). These technical reports show that light-dependent CRY-CIB interaction has many different utilities in biomedical research and drug discovery, including the control of transcription, protein translocation, protein trafficking, lipid metabolism, drug delivery, enzyme inactivation, and any other biochemical reactions that require, are associated with, or can be regulated by protein-protein interactions.

Following the discovery of the light-dependent CRY-CIB interaction and the utilities associated with this phenomena, there is a need for methods and materials that can be used to modulate the CRY-CIB interaction in biomedical applications adapted to harness and utilize this light-dependent CRY-CIB interaction.

### SUMMARY OF THE INVENTION

Building upon the discovery of blue light-dependent cryptochrome (CRY) dimerization/oligomerization, we have identified important genes/proteins which are Blue-light Inhibitors (“BICs”) of cryptochromes 1 and 2 (CRY1 and CRY2). As discussed in detail below, we have discovered that BICs can inhibit light-dependent CRY dimerization, CRY phosphorylation, and all physiological functions of cryptochromes. Importantly, the Arabidopsis BICs used in the studies below not only inhibit the function and oligomerization in cryptochromes plant cells, but also show activity in a variety of other cells including mammalian cells. In illustrative working embodiments of the invention, these genes/proteins are BIC1 (e.g. Arabidopsis locus AT3G52740) and BIC2 (e.g. Arabidopsis locus AT3G44450). Surprisingly, these Arabidopsis BICs are observed to inhibit light-dependent dimerization/oligomerization of plant cryptochromes in the human embryo kidney cell line HEK293. The unexpected function and associated versatility of these genes/proteins in such vastly different biological systems makes the invention highly useful in a broad range of biomedical applications.

Illustrative systems and methods described herein utilize BICs to suppress blue light-dependent dimerization of CRY, the physical interactions of CRY with its signaling partners such as cryptochrome-interacting basic helix-loop-helix1 protein (CIB), and/or physiological activities of the photoreceptor. These systems and methods allow for the control of optogenetics reactions such as light-induced regulation of transcription, protein translocation, DNA recombination, phosphoinositide metabolism, epigenetics change, and reversible protein inactivation traps. In addition, the BICs described herein may be used to inhibit, suppress, reverse or otherwise control the strength of any reaction dependent on or associated with the blue light-dependent protein interaction between a CRY protein and a CRY-signaling protein (e.g. CRY2-CIB1 interaction).

The invention disclosed herein has a number of embodiments including compositions, methods and systems that utilize BIC genes/proteins (and CRY and CIB genes/proteins) and the associated discoveries relating to their function. An illustrative embodiment of the invention is a composition of matter comprising a polynucleotide encoding a blue-light inhibitor of cryptochrome (BIC) polypeptide that inhibits the light dependent function of a cryptochrome polypeptide, and which is covalently linked to a heterologous promoter that controls the expression of the BIC gene. For example, the polynucleotide encoding the blue-light inhibitor of cryptochrome (BIC) polypeptide can be disposed within a plasmid and operably linked to an inducible promoter; and/or a promoter selected for its ability to regulate gene/protein expression in a particular type of organism or cell lineage. Typically in such embodiments, the BIC polypeptide inhibits blue-light dependent dimerization of cryptochrome 2 polypeptide (SEQ ID NO: 6), and the BIC polypeptide has an at least 90% amino acid sequence identity to SEQ ID NO: 2 or SEQ ID NO: 4. Optionally, the BIC polypeptide is coupled to a heterologous amino acid segment such as a fusion protein or a peptide tag.

Another embodiment of the invention is a composition of matter comprising a cryptochrome (CRY) polypeptide, a cryptochrome polypeptide-interacting basic helix-loop-helix (CIB) polypeptide; and a blue-light inhibitor of cryptochrome (BIC) polypeptide; wherein at least one of these three polypeptides of is coupled to a heterologous amino acid segment such as a fusion protein or a peptide tag.

Another embodiment of the invention is a method for modulating a reaction between a cryptochrome (CRY) protein and a cryptochrome polypeptide-interacting basic helix-loop-helix (CIB) protein. This method comprises combining a CRY protein and a CIB1 protein with a blue-light inhibitor of cryptochrome (BIC) protein, wherein the BIC protein is a BIC1 protein or a BIC2 protein; and the BIC protein inhibits, suppresses or reverses the blue light-dependent interaction between the CRY2 protein and the CIB1 protein. Typically in these embodiments, the CRY polypeptide has an at least 90% amino acid sequence identity to SEQ ID NO: 6; the CIB polypeptide has an at least 90% amino acid sequence identity to SEQ ID NO: 8; and/or the BIC polypeptide has an at least 90% amino acid sequence identity to SEQ ID NO: 2 or SEQ ID NO: 4.

Yet another embodiment of the invention is an optogenetic system including a vessel comprising one or more compartments containing a cryptochrome (CRY) protein, a cryptochrome polypeptide-interacting basic helix-loop-helix (CIB) protein, and a blue-light inhibitor of cryptochrome (BIC) protein. This optogenetic system embodiment of the invention further includes a blue light source. Typically in these embodiments, the CRY polypeptide has an at least 90% amino acid sequence identity to SEQ ID NO: 6; the CIB polypeptide has an at least 90% amino acid sequence identity to SEQ ID NO: 8; and/or the BIC polypeptide has an at least 90% amino acid sequence identity to SEQ ID NO: 2 or SEQ ID NO: 4. In certain embodiments, the system further comprises a cell culture media, for example one used to culture bacterial cells or one used to culture yeast cells or one used to culture plant cells or one used to culture mammalian cells.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 provides data showing that BIC1 inhibits light-dependent CRY2-CIB1 interaction in HEK293 cells. HEK293 cells co-expressing Flag-CRY2, GFP-CIB1, in the presence of absence of Myc-BIC1 fusion proteins were subjected to co-immunoprecipitation analysis. The immunoprecipitation reactions using anti-FLAG antibody were analyzed by immunoblot assay probed with the anti-Flag antibody (CRY2), anti-Myc antibody (BIC1), or the anti-GFP antibody (CIB1).

FIG. 2 provides data showing that BIC1 and BIC2 inhibit the function of CRY1 and CRY2. (FIG. 2A) The representative hypocotyl image of the WT, bic1bic2, bic1D-1, BIC2-GFP and cry1cry2 grown in blue light (4 μmol m⁻² s⁻¹) and in the dark for 5 days. (FIG. 2B) Hypocotyl length of each indicated genotype grown in blue light (0 to 100 μmol m⁻² s⁻¹) for 5 days. (FIG. 2C) Hypocotyl length of each indicated genotype grown in dark, blue light (10 μmol m⁻²s⁻¹), red light (10 μmol m⁻² s⁻¹) or far-red light (5 μmol m⁻² s⁻¹) for 5 days. (FIG. 2D) Flowering phenotype of each genotype grown in long day conditions (16 hours light, 8 hours dark) for 31 days. (FIGS. 2E and F) The time to flowering and the number of rosette leaves at the time of flowering of the indicated genotypes shown in FIG. 2D. (FIG. 2G) quantitative PCR (qPCR) showing mRNA expression of FT gene in the seedlings of each genotype grown in long day condition for 10 days. (FIGS. 2H and I) qPCR showing mRNA expression of BIC1 (FIG. 2H) or BIC2 (FIG. 2I) in the samples indicated. The etiolated seedlings were irradiated with blue light (20 μmol m⁻² s⁻¹) for the durations indicated. The relative expression unit (REU) were calculated by normalization of the mRNA signal in each sample with that in WT grown in dark;

FIG. 3 provides data from kinetics analysis showing BIC1 or BIC2 inhibits the phosphorylation of CRY1 or CRY2, or the degradation of CRY2 in response to blue light. (FIGS. 3A, B, E and H) Immunoblots of sample prepared from 7-day-old etiolated seedling expose to blue light (31±2 μmol m⁻²s⁻¹) and probed with antibody to CRY1 (FIGS. 3A and B) or CRY2 (FIGS. 3E and H). The membranes were striped and probed with antibody to HSP for loading control. (FIGS. 3C, D, F and I) The relative band intensities of phosphorylated CRY1 (FIGS. 3C and D) or CRY2 (FIGS. 3F and I) were presented as CRY1Pi/CRY1 or CRY2pi/CRY2. (FIGS. 3G and J) The relative band intensities of CRY2 in the seedling of bic1-1D (FIG. 3G) or BIC2-GFP (FIG. 3J) treated with blue light for the duration indicated were presented as CRY2^{B}/CRY2^{D};

FIG. 4 provides data showing redox-dependent CRY dimerization, and blue-light-induced CRY and BIC interaction. (FIG. 4A) The β-galactosidase (β-gal) assay showing the blue-light-dependent formation of CRY2 dimer. Yeast cells were kept in darkness (D) or illuminated with blue light (B5, 5 μmol m⁻² s⁻¹; B25, 25 μmol m⁻² s⁻¹; B50, 50 μmol m⁻² s⁻¹) for the indicated time. (FIG. 4B) Immunoblot showing the blue-light-induced dimerization of CRY2 expressed in HEK293FT (H293) cells. The cells were lysed, divided into 12 samples and irradiated by blue light (40 μmol m⁻²s⁻¹) for the indicated durations. For β-mercaptoethanol (2-Me) treatment, 6 samples were added with 2-Me to 5% (v/v), and then all the samples were analyzed by western blot using anti-CRY2 antibody. CRY2, monomeric CRY2; (CRY2)2, CRY2 dimer; (CRY2)ₙ, CRY2 oligomer. (FIG. 4C) The H293 cell lysate were prepared in dark, irradiated with blue light for the durations indicated. For the NEM treatment, the samples were added with NEM (50 mM), and then the samples were analyzed by western blot using anti-CRY2 antibody. (FIGS. 4D and E) Dimerization and oligomerization of CRY2 proteins are blue light-induced in vivo. Long day-grown seedlings of CRY2-OX line H3 were kept under continuous red light (25 μmol m⁻² s⁻¹) for two days before irradiated with 55 μmol m⁻² s⁻¹ Blue or Red light for different times (FIG. 4D), or irradiated with blue or red light with different fluence rates for 1 hour (FIG. 4E). Nuclear proteins extracted from each samples were used for western blot analysis with anti-CRY2 antibody. Histone H3 probed with anti-H3 antibody was used as a loading control. (FIG. 4F) β-Gal assay of yeast cells expressing indicated proteins kept in darkness or irradiated with blue light (50 μmol m⁻² s⁻¹) for 2 hours. (FIG. 4G) BiFC assay shown the interaction between CRY2 and BIC2. The percentage of protoplasts that showed BiFC fluorescence signals was counted. Each sample contains at least 30 protoplast. Means and SD (n=3) are shown;

FIG. 5 provides data showing that BIC interacts with CRY2 to inhibit redox-dependent CRY2 dimerization and function. (FIG. 5A) BIC1 inhibits the dimerization of CRY2 expressed in H293 cells. The H293 cells were transfected with vector expressing CRY2 only (CRY2), or together with vector expression Myc-BIC1 (CRY2+BIC1) and cultured in dark for 24 hours. The cells were cracked and divided into equally 12 tubes, then treated with blue light (40 μmol m⁻² s⁻¹) for the duration indicated. The sample were analyzed by western blot using anti-CRY2 antibody and anti-Myc antibody sequentially. (FIG. 5B) β-Gal assay of yeast cells expressing indicated proteins kept in darkness (D) or irradiated with blue light (B50, 50 μmol m⁻² s⁻¹) for indicated durations. (FIG. 5C) Fluorescence images showing the formation of CRY2-GFP nuclear bodies in the protoplasts of WT, BIC1-OX line or BIC-OX line. The protoplasts transformed with CRY2-GFP construct were kept in darkness overnight and then irradiated with blue light (20 μmol m⁻² s⁻¹) for the time indicated. (FIG. 5D) The percentage of protoplasts showed the formation of CRY2-GFP nuclear bodies were counted. Each sample contains at least 50 protoplast. Means and SD (n=3) are shown. (FIG. 5E) BiFC analysis of the blue-light-induced formation of CRY2 nuclear bodies in protoplasts of WT, BIC1-OX line or BIC2-OX line. The protoplasts transformed with cYFP-CRY2 and nYFP-CRY2 constructs were kept in darkness overnight and then irradiated with blue light (20 μmol m⁻² s⁻¹) for the time indicated. (FIG. 5F) The percentage of protoplasts that showed BiFC fluorescence signals in the form of nuclear body as in (FIG. 5E) were counted. Each sample contains at least 50 protoplast. Means and SD (n=3) are shown. (FIG. 5G) BIC1 inhibits the dimerization and oligomerization of CRY2 proteins in plant cells treated with blue light. Long day-grown seedlings of CRY2-OX line (BIC −) or CRY2-OX/BIC1-OX line (BIC +) were kept in continuous red light (25 μmol m⁻² s⁻¹) for two days before irradiated with 55 μmol m⁻² s⁻¹ Blue or Red light for 1 hour. Nuclear proteins extracted from each samples were used for western blot analysis with anti-CRY2 antibody. Histone H3 probed with anti-H3 antibody was used as loading control. (FIG. 5H) The 3-gal assay showing the interaction between CRY2 and SPA1, or between CRY2 and CIB1, with/without BIC1 or BIC2 as Bait mate respectively. Yeast cells were kept in dark or illuminated with blue light (B20, 20 μmol m⁻² s⁻¹);

FIG. 6 provides a schematic model of CRY-BIC circuitry;

FIG. 7 provides data showing that CRY2-CIB1 mediates blue light control of transcription in zebrafish embryo. Relative reporter gene transcription activity (LUC/REN) was measured under conditions indicated in the absence or presence of effectors (CIB1 or CRY2) and blue light (Dark or Blue);

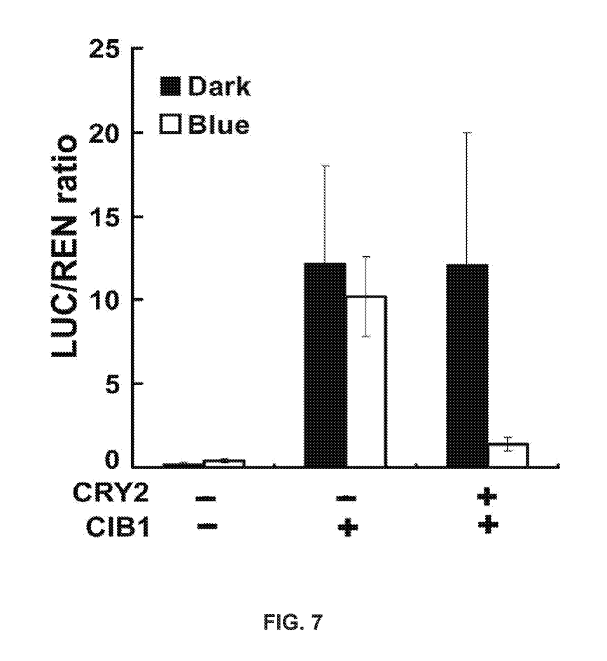

FIG. 8 provides data showing that light-dependent dimerization/oligomerization of human CRY (HsCRY2), and BIC2-dependent inhibition of hsCRY2 dimerization/oligomerization. HEK293 cells expressing HsCRY2 in the absence (left) or presence (right) of Arabidopsis BIC2 were illuminated with blue light for the time indicated (bottom). The light-dependent HsCRY2 dimerization (HsCRY2)2 or oligomerization (HsCRY2)ₙ (left panel) and BIC2-dependent inhibition of HsCRY2 dimerization/oligomerization are shown.

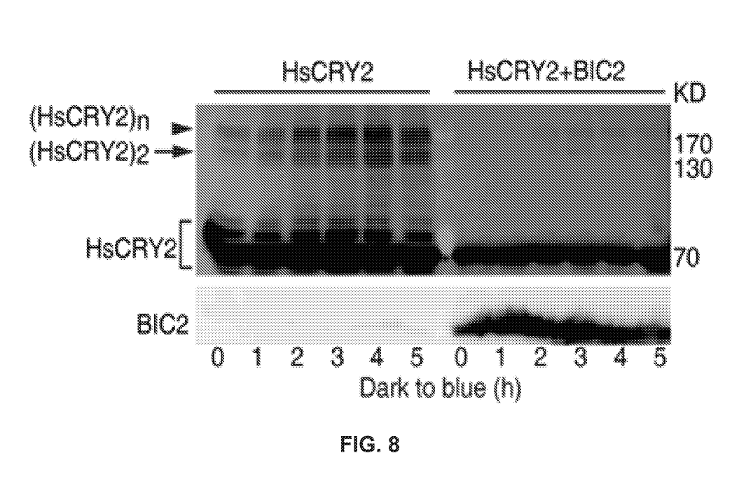

FIG. 9 provides data showing that BIC1 inhibits blue-light dependent Arabidopsis CRY2 dimerization. FIG. 9A shows blue light-dependent CRY2 dimerization in Arabidopsis. 7-day old etiolated seedlings coexpressing Myc-CRY2 and GFP-CRY2 were exposed to 30 μmol m⁻² s⁻¹ blue light for 20 sec (0.33 min), 40 sec (0.67 min), 1 min, 2 min, 5 min and 10 minutes. GFP-Trap-A were used to immunoprecipitate GFP-CRY2. GFP-CRY2 (IP signal) and Myc-CRY2 (co-IP signal) were detected by GFP or Myc antibody, respectively. FIG. 9B shows quantitative Co-IP analyses of CRY2 photodimerization in HEK293T cells. HEK293T cells co-expressing FLUC-CRY2 and REN-CRY2 were exposed to blue light (30 μmol·m⁻²·s⁻¹) for the time indicated, lysed, aliquots removed for the measurement of ATL (Adjusted Total Luminescence), and FLUC-CRY2 precipitated by anti-Flag antibody conjugated beads. Photodimerization of CRY2 was quantified by the dual-luciferase assay to calculate the Normalized Dimerization Ratio (NDR) by the formula: NDR=[(REN/LUC)L/(REN/LUC)D]/ATL, where LUC is the LUC luminescence detected for the precipitated FLUC-CRY2, REN is the REN luminescence detected for the co-precipitated REN-CRY2. (REN/LUC)L or (REN/LUC)D were measured in cells exposed to light or darkness, respectively. ATL (Adjusted Total Luminescence) is the cell volume-adjusted sum of LUC and REN luminescence of the cell lysates before immunoprecipitation, in which REN luminescence is converted to the LUC equivalent by the standard curve prepared by analyses of the LUC-REN fusion protein (not shown). ATL represents concentration of the CRY2 protein. Regression analysis of NDR as the function of time, SD (n=3), D1/2, and a correlation analysis (insert) of the results from two qCo-IP experiments under the same conditions are shown. The Maximum Photodimerization (Dm=4.7048) is defined by the maximum NDR; the Rate of Photodimerization (D1/2=1 min) is defined by the time needed to reach Dm. FIG. 9C shows HEK293T cells coexpressing Flag-CRY2, Myc-CRY2 and GFP-BIC1 or GFP were exposed to 180 μmol m⁻² s⁻¹ blue light for the time indicated. Anti-Flag antibody conjugated beads were used to perform the immunoprecipitations. Flag-CRY2 (IP signal) and Myc-CRY2 or GFP-BIC1 (co-IP signals) were detected by Flag and Myc or GFP antibody, respectively.

FIG. 10 provides data showing dimerization activity of CRY from different organisms. In this Figure, Co-immunoprecipitation assay of cryptochromes of the indicated organisms Arabidopsis, Rice (Oryza sativa or Os), Soybean (Glycine Max or Gm), Zebrafish (Danio rerio or Z), Monarch Butterfly (Danaus plexippus or Dp), and Human. HEK293T cells co-expressing Flag-CRY and myc-CRY were exposed to 100 mol m⁻² s⁻¹ blue light for 2 hours (+) or kept in the dark (−). Anti-Flag antibody conjugated beads were used to perform the immunoprecipitations. Flag-CRY (IP signal) and Myc-CRY (co-IP signal) were detected by anti-Flag or anti-Myc antibody respectively.

FIG. 11 provides data showing that Arabidopsis BIC1 interacts with both human CRY1 and CRY2, however the interactions have no effect on the dimerization activity of human CRYs.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

All publications, patents, and patent applications cited herein are hereby incorporated by reference in their entirety for all purposes. For example, U.S. provisional patent application No. 61/112,033, titled “A CRY/CIB1-REGULATED GENE EXPRESSION SYSTEM IN YEAST AND HUMAN CELLS”, filed Nov. 6, 2008; U.S. provisional patent application No. 61/258,415, titled “A CRY/CIB1-REGULATED GENE EXPRESSION SYSTEM IN YEAST AND HUMAN CELLS”, filed Nov. 5, 2009; and Liu et al. Science 322.5907 (2008): 1535-1539; Wang et al., Science 21 Oct. 2016: Vol. 354, Issue 6310, pp. 343-347; Meng et al., Plant Cell. 2013 November; 25(11):4405-20 Epub 2013 Nov. 22; and Liu et al., Trends Plant Sci. 2011 December; 16(12):684-91 Epub 2011, are incorporated herein by reference.

Cryptochromes (CRYs) are photolyase-like flavoproteins that act as blue light receptor in plants and animals. There are two types of CRYs: type 1 CRY are photoreceptors in plants and animals, whereas type 2 CRYs act as light-independent transcription regulator and core components of the circadian clock in animals, including human. It has been previously shown that Arabidopsis CRY2 undergoes blue light-dependent dimerization, referred to as photodimerization, to become physiologically active, that Arabidopsis BIC1 and BIC2 proteins interact with Arabidopsis CRY2 to inhibit photodimerization and all biochemical and physiological activities of CRY2, and that human CRY2 also undergo homodimerization (Science 2016, 354:343-347).

Blue-light inhibitors of CRY (BICs) are the first proteins known to possess the activity to regulate the light-dependent protein interaction between a CRY protein and a CRY-signaling protein such as cryptochrome-interacting basic helix-loop-helix1 protein (CIB), (e.g. CRY2-CIB1 interaction). CRYs regulate light responses by interacting with CRY-signaling partners, such as CIBs (Cryptochrome-interacting bHLHs) and COP1/SPA (Constitutive phoyomorphogenic 1/Suppressor of PhyA-105) to control blue light-responsive gene expression changes and photomorphogenesis. The desensitization mechanism and BICs described herein have not been previously known in the art.

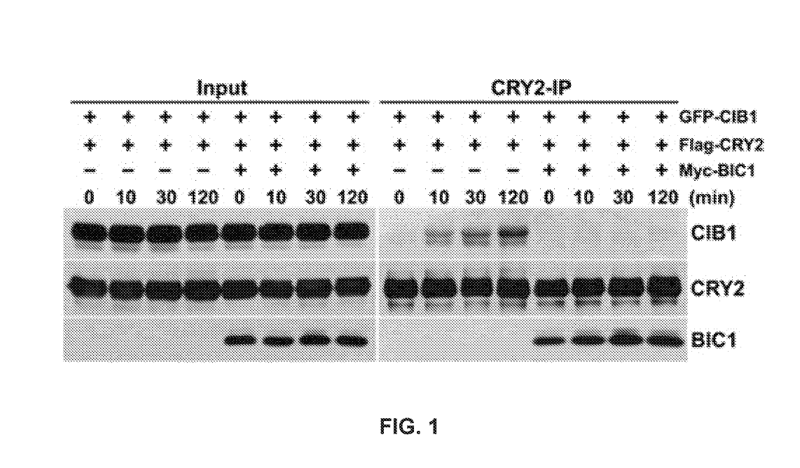

As illustrative embodiments of the invention, two related genes/proteins of Arabidopsis are described herein, referred to as BIC1 (AT3G52740; SEQ ID NO: 2) and BIC2 (AT3G44450; SEQ ID NO: 4), which stand for Blue-light Inhibitors of CRY1 and 2. BICs (e.g. BIC1 and BIC2) inhibit light-dependent CRY dimerization, CRY phosphorylation, and all physiological functions of cryptochromes. Importantly, the Arabidopsis BICs not only inhibit the function and oligomerization in cryptochromes plant cells, they also have demonstrated activities in human cells, namely Arabidopsis BICs inhibit light-dependent dimerization/oligomerization of plant cryptochromes in the human embryo kidney cell line HEK293 (FIG. 1).

Without being limited to a particular theory, a CRY-BIC negative feedback model is provided to explain the photoactivation and inactivation mechanisms of plant cryptochromes. According to this model, cryptochromes exist as inactive monomers in the absence of light. In response to blue light, photoexcited cryptochromes form active homodimers or oligomers that interact with CRY-signaling proteins to activate gene expression changes responsible for photomorphogenesis as well as accumulation of the BIC proteins. The BIC proteins interact with cryptochromes to monomerize and inactivate the photoreceptors, resulting in homeostasis of the active cryptochromes and sustainability of cellular photosensitivity.

As shown herein, BIC can be used as a potent inhibitor for any study that employs the blue light-dependent CRY2-CIB interaction. Thus, BICs can be effective regulators of any optogenetics method that relies on the light-dependent protein interaction between a CRY protein and a CRY-signaling protein (e.g. CRY2-CIB1 interaction). Such optogenetics tools are widely used in the study of cellular and molecular mechanisms underlying human diseases and in drug discoveries, especially for neural diseases. Because human CRY1 and CRY2 undergo light-independent interaction, this allows us to manipulate human circadian clock in cells or tissues. Because the circadian clock affect many human diseases, including cancer and diabetes, assays based upon this interaction can be used for drug discovery. Moreover, as Arabidopsis BIC1 and BIC2 interact with human CRY1 or CRY2, embodiments of the invention can also be used in the manipulation of human circadian clock in cells or tissues.

Because the CRY protein and CRY-signaling protein interaction is dependent on CRY dimerization/oligomerization, including BICs in any optogenetics reaction dependent on the CRY protein and CRY-signaling protein interaction has the advantage of providing previously unknown control of such reactions. BICs are the first proteins discovered to inhibit CRY dimerization/oligomerization and CRY protein and CRY-signaling protein interaction. Therefore, there is presently no similar/competing technology in the art for the control of CRY dimerization/oligomerization and CRY protein and CRY-signaling protein interaction.

In an illustrative working example, Arabidopsis CIB1 (cryptochrome-interacting basic-helix-loop-helix) protein has been identified to interact with CRY2 (cryptochrome 2) in a blue light-specific manner in yeast and Arabidopsis cells. Light-dependent CRY2-CIB1 interaction has been utilized as an optogenetics tool to achieve light-induced regulation of transcription, protein translocation, DNA recombination, phosphoinositide metabolism, epigenetics change, and reversible protein inactivation trap. The use of BICs to inhibit this light-dependent CRY2-CIB1 interaction thus allows for the modulation/control of all these and other optogenetics reactions based on the CRY-CIB interaction.

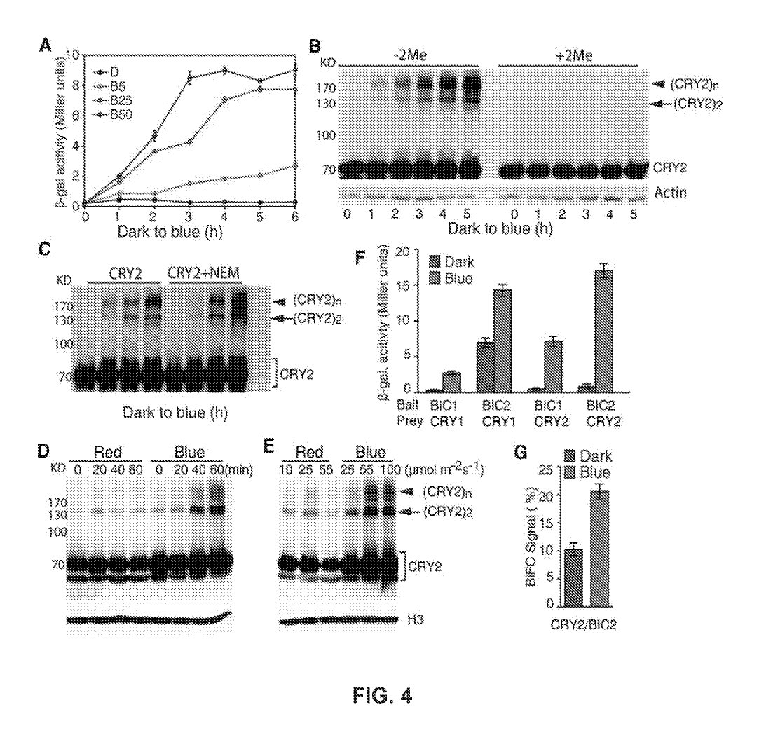

Additionally, CRY is a critical component of the human circadian clock, which is associated with numerous human diseases, including diabetes, obesity, cancer, mania, etc. Because human CRYs also undergo dimerization/oligomerization, the fact that BICs directly inhibit dimerization/oligomerization of human cryptochromes in human cells (FIG. 4A) provides a novel technology for regulating CRY and clock activity in human cells, affecting the treatment of various human diseases. Many proteins are known to affect the activity of human CRY and clock, including PER, CLOCK, BMAL, FBX3, FBX21, CKI, SETX, SIN3A, etc. These proteins can be used to develop technologies for regulating CRY and clock activity. A further advantage of BIC in biomedicine research or drug discovery is that the human genome does not encode proteins related to BICs. Therefore, use of the novel plant BIC proteins described herein offers specificity not found in any potential technology dependent on the above-mentioned human proteins (i.e. PER, CLOCK, BMAL, etc.). Further, BICs inhibit the function of plant cryptochromes, and light-dependent growth and reproduction. Thus, in certain embodiments, BICs can be used to regulate crop growth and reproduction as well as crop yield.

The invention disclosed herein has a number of embodiments including compositions, methods and systems that utilize BIC genes/proteins and the associated discoveries relating to its function. An illustrative embodiment of the invention is a composition of matter comprising a polynucleotide encoding a blue-light inhibitor of cryptochrome (BIC) polypeptide that inhibits the light dependent function of a cryptochrome polypeptide, and which is coupled to a heterologous promoter that controls the expression of the BIC gene. As used herein, promoter simply refers to a region of DNA that initiates transcription of a particular gene such as CRY, BIC or CIB. Promoters are typically about 100-1000 base pairs in length. As used in this context, “heterologous” simply means a promoter that is different from the promoter found in the wild type gene.

For example, the polynucleotide encoding the blue-light inhibitor of cryptochrome (BIC) polypeptide can be disposed within a plasmid and operably linked to an inducible promoter; and/or a promoter selected for its ability to regulate gene/protein expression in a particular type of organism or cell lineage. Optionally, the BIC polypeptide in the composition is coupled to a heterologous amino acid segment such as a fusion protein or a peptide tag. Protein or peptide tags are non-naturally occurring amino acid sequences that coupled onto a protein sequence such as CRY, BIC or CIB (e.g. by creating a polynucleotide that encodes CRY, BIC or CIB fused in reading frame to an amino acid sequence that that is different from the amino acid sequence found in the wild type protein. Affinity tags are appended to proteins so that they can be purified from their crude biological source using an affinity technique. These include chitin binding protein (CBP), maltose binding protein (MBP), and glutathione-S-transferase (GST). The poly(His) tag is a widely used protein tag; it binds to metal matrices. Chromatography tags are used to alter chromatographic properties of the protein to afford different resolution across a particular separation technique. Often, these consist of polyanionic amino acids, such as FLAG-tag.

In certain embodiments of the invention, the polynucleotide encoding the BIC polypeptide is a transgene that expresses the BIC polypeptide within a cell. Optionally this cell is a plant cell or a mammalian cell. These compositions can include additional genes or proteins, for example a polynucleotide encoding a cryptochrome and/or CIB polypeptide.

Typically in embodiments of the invention, the BIC polypeptide inhibits blue-light dependent dimerization of cryptochrome 2 polypeptide (SEQ ID NO: 6), and the BIC polypeptide has an at least 90% or 95% amino acid sequence identity to SEQ ID NO: 2 or SEQ ID NO: 4 (e.g. using BLAST or ClustalW algorithms). Optionally in embodiments of the invention using multiple polypeptides, the BIC polypeptide has an at least 90% amino acid sequence identity to SEQ ID NO: 2 or SEQ ID NO: 4; the CIB polypeptide has an at least 90% amino acid sequence identity to SEQ ID NO: 8; and/or the CRY polypeptide has an at least 90% amino acid sequence identity to SEQ ID NO: 6.

Another embodiment of the invention is a composition of matter comprising a cryptochrome (CRY) polypeptide a cryptochrome polypeptide-interacting basic helix-loop-helix (CIB) polypeptide; and a blue-light inhibitor of cryptochrome (BIC) polypeptide; wherein at least one (or two or three) of these three polypeptides of is coupled to a heterologous amino acid segment such as a fusion protein or a peptide tag. Such embodiments of the invention can further include Flavin adenine dinucleotide (FAD). In some embodiments of the invention, the composition is disposed in an in vitro environment. Optionally, the composition is disposed within a mammalian cell.

Another embodiment of the invention is a method for modulating a reaction between a cryptochrome (CRY) protein and a cryptochrome polypeptide-interacting basic helix-loop-helix (CIB) protein. This method comprises combining a CRY protein and a CIB1 protein with a blue-light inhibitor of cryptochrome (BIC) protein, wherein the BIC protein is a BIC1 protein or a BIC2 protein; and the BIC protein inhibits, suppresses or reverses the blue light-dependent interaction between the CRY2 protein and the CIB1 protein. Typically in these embodiments, the CRY polypeptide has an at least 90% amino acid sequence identity to SEQ ID NO: 6; the CIB polypeptide has an at least 90% amino acid sequence identity to SEQ ID NO: 8; and/or the BIC polypeptide has an at least 90% amino acid sequence identity to SEQ ID NO: 2 or SEQ ID NO: 4

Yet another embodiment of the invention is an optogenetic system comprising a vessel comprising one or more compartments containing a cryptochrome (CRY) protein, a cryptochrome polypeptide-interacting basic helix-loop-helix (CIB) protein, and a blue-light inhibitor of cryptochrome (BIC) protein. This optogenetic system embodiment of the invention further includes an aqueous solution disposed within the vessel; and a blue light source. Typically in these embodiments, the CRY polypeptide has an at least 90% amino acid sequence identity to SEQ ID NO: 6; the CIB polypeptide has an at least 90% amino acid sequence identity to SEQ ID NO: 8; and/or the BIC polypeptide has an at least 90% amino acid sequence identity to SEQ ID NO: 2 or SEQ ID NO: 4. In certain embodiments, the system further comprises a cell culture media, for example one used to culture bacterial cells or one used to culture yeast cells or one used to culture plant cells or one used to culture mammalian cells. Optionally, the system further comprises bacterial or yeast or plant or mammalian cells.

As noted above, embodiments of the invention include methods for modulating a reaction dependent on a blue light-dependent protein interaction between a CRY protein and a CRY-signaling protein. The method comprises combining a CRY protein and a CRY-signaling protein with a BIC protein, wherein the BIC protein modulates the blue light-dependent interaction between the CRY protein and the CRY-signaling protein. Other embodiments of the invention include compositions of matter comprising a CRY protein, a CRY-signaling protein, and a BIC protein, wherein the BIC protein modulates a blue light-dependent interaction between the CRY protein and the CRY-signaling protein.

In various embodiments, the BIC protein modulates by inhibiting, suppressing or reversing the blue light-dependent interaction between the CRY protein and the CRY-signaling protein. Specifically, the BIC protein binds to the CRY protein to suppress blue light-dependent dimerization of CRY, CRY phosphorylation, the physical interactions of CRY with its signaling partners, and/or physiological activities of the photoreceptor. In one instance, the CRY protein is a CRY2 protein (SEQ ID NO: 6) and the CRY-signaling protein is a CRY2-signaling protein (e.g. CIB1, SEQ ID NO: 8). The BIC protein is a BIC1 protein (SEQ ID NO: 2) or a BIC2 protein (SEQ ID NO: 4).

Further aspects and embodiments of the invention are disclosed in the following example:

### EXAMPLES

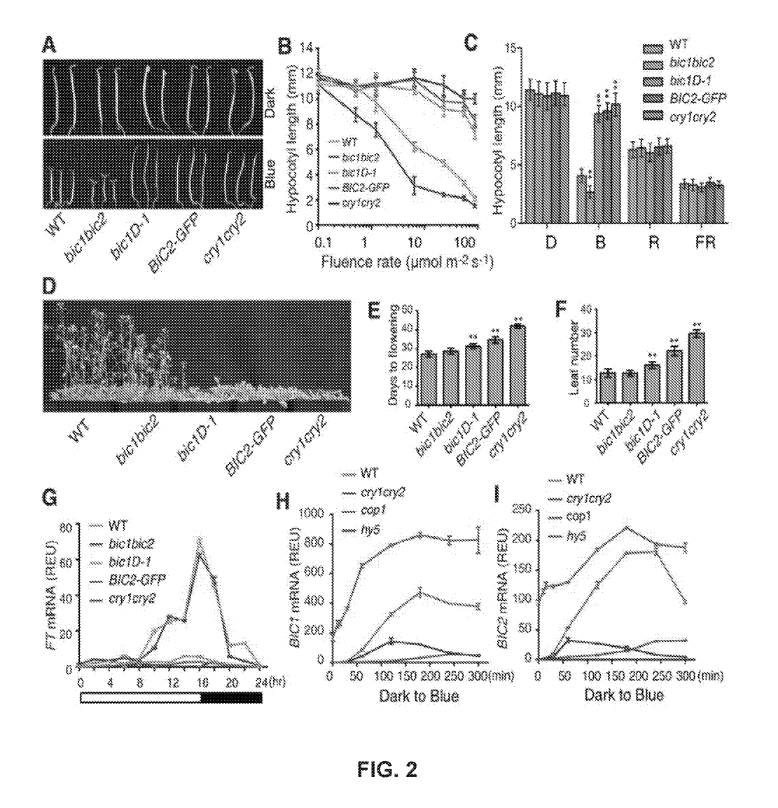

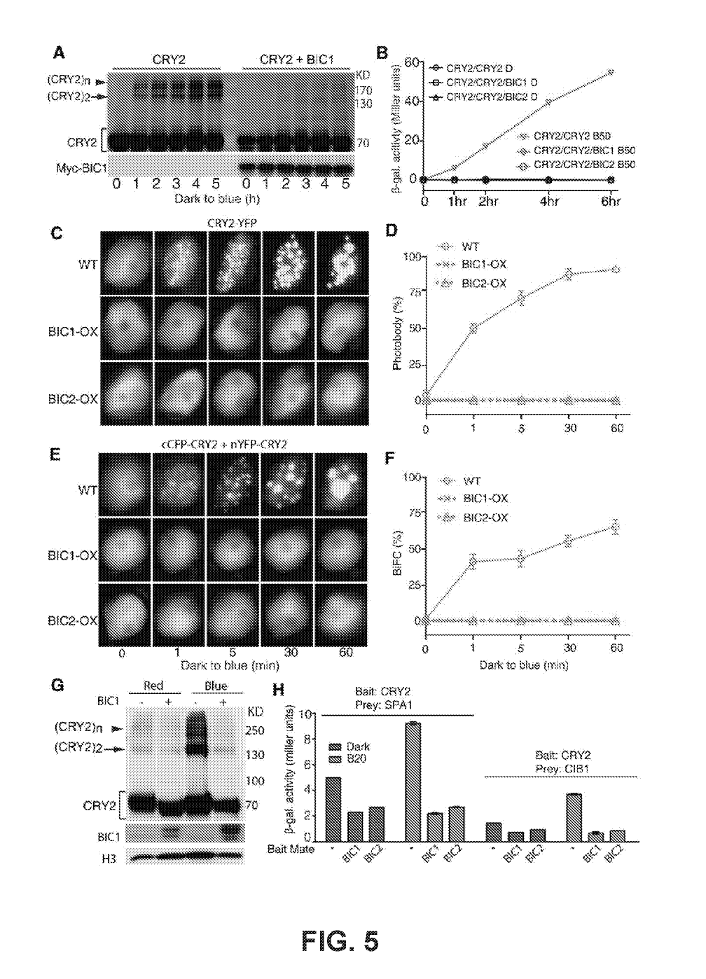

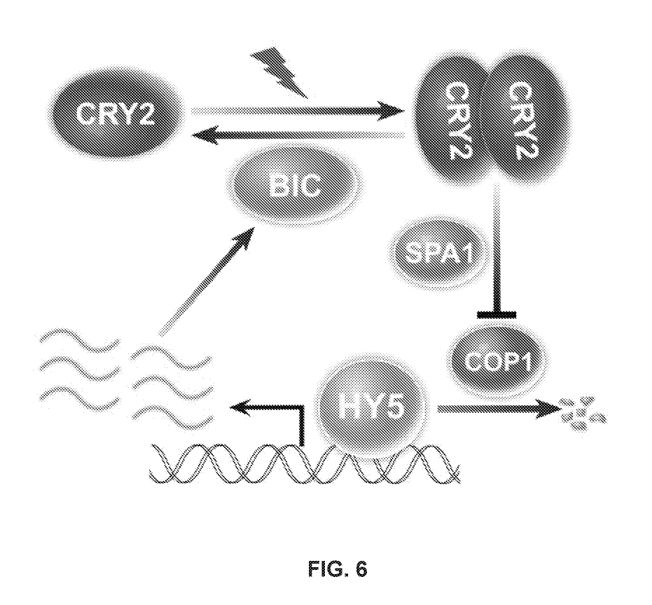

As noted above, cryptochromes are blue-light receptors of the circadian clock in animals and photomorphogenesis in plants, but the photochemical mechanism underlying cryptochrome desensitization remain unknown. It has been found that Arabidopsis cryptochrome 2 (CRY2) undergoes blue light-dependent dimerization via disulfide bonds, resulting in activation of the photoreceptor. Two novel regulators of cryptochromes have been identified, referred to as Blue-light Inhibitors of Cryptochromes 1 and 2 (BIC1 and BIC2), which abolish all blue light-dependent activities of cryptochromes tested, including physiological activities, light-dependent phosphorylation, photobody formation, and degradation of cryptochromes. As demonstrated herein, the BIC proteins interact with CRY2 to inhibit dimerization and oligomerization of photoexcited CRY2, whereas cryptochromes mediate blue-light activation of expression of the BIC genes. These results support a hypothesis that the photoexcited cryptochromes are oxidized to form active dimers, leading to signal transduction and expression of the BIC genes, which in return interact and desensitize the photoactive photoreceptors. See, FIGS. 2-6.

### Example 1: Photoactivation and Inactivation of Arabidopsis Cryptochrome 2

Aspects of the invention discussed in this example are disclosed in Wang et al., Science 21 Oct. 2016: Vol. 354, Issue 6310, pp. 343-347 (referred to as “Wang et al.” in this Example). The references cited in this example are those found at the end of this example disclosure.

The Arabidopsis genome encodes two cryptochromes (CRYs), CRY1 and CRY2, which act as photoreceptors mediating blue-light inhibition of hypocotyl elongation and long-day (LD) stimulation of floral initiation (1-4). CRYs regulate light responses by interacting with CRY signaling partners, such as CIBs (cryptochrome interacting basic helix-loop-helixes) and COP1/SPA (constitutive photomorphogenic 1/suppressor of PhyA-105), to regulate blue light-responsive gene expression changes and photophysiology responses (5-7). Homodimers are the physiologically active form of plant CRYs, but it has remained unclear how light affects CRY dimerization or photoactivation (8, 9). Photoactivated CRYs are also expected to undergo inactivation to maintain sustainable photosensitivity of the cell, which may be accomplished by thermal relaxation or other mechanisms (10).

We reasoned that identification of possible negative regulators of CRYs may help to elucidate the photoactivation and inactivation mechanisms of CRYs. We therefore screened for such genes in the Arabidopsis FOX (full-length cDNA overexpressing gene hunting system) library, which contains transgenic lines individually overexpressing about 10,000 independent full-length Arabidopsis cDNAs (11). We identified multiple FOX lines (bic1D-1, bic1D-2, and bic1D-3) that overexpress the same gene and exhibit similar phenotypes resembling that of the cry1cry2 mutant (12), including blue light-insensitive hypocotyl growth, reduced blue-light stimulation of anthocyanin accumulation and gene expression, and delayed floral initiation in LD photoperiod (FIG. 1 and FIGS. S1 and S2 of Wang et al). The corresponding FOX gene was identified and referred to as BIC1 (Blue-light Inhibitor of Cryptochromes 1, At3G52740), which has an Arabidopsis homolog referred to as BIC2 (At3G44450) (FIG. S3 of Wang et al). BIC1 and BIC2 appear to be nuclear proteins (FIG. S4 of Wang et al).

Independent transgenic lines overexpressing various BIC fusion proteins under control of either the constitutive promoters or the respective BIC promoters recapitulated the light-insensitive phenotypes of the BIC1-overexpressing FOX lines and the cry1cry2 mutant (FIG. 1 and FIG. S1 of Wang et al). The bic1 and bic2 monogenic mutants showed no obvious phenotypic alterations, whereas the bic1bic2 double mutant and the BIC RNA interference lines exhibited phenotypes mimicking that of the CRY-overexpressing plants (FIG. 1 and FIGS. S2, S5, and S6 of Wang et al), which suggests that BIC1 and BIC2 act redundantly to inhibit the function of CRYs. Analyses of the genetic interactions between the BIC and CRY genes support this hypothesis (FIG. S7 of Wang et al): Neither bic1bic2 mutation nor BIC overexpression altered the blue light-insensitive phenotypes of the cry 1cry2 mutant (FIG. S7, D to F of Wang et al), whereas overexpression of BIC1 or BIC2 effectively suppressed the blue light-hypersensitive phenotype of plants overexpressing CRY2 (FIG. S7G of Wang et al).

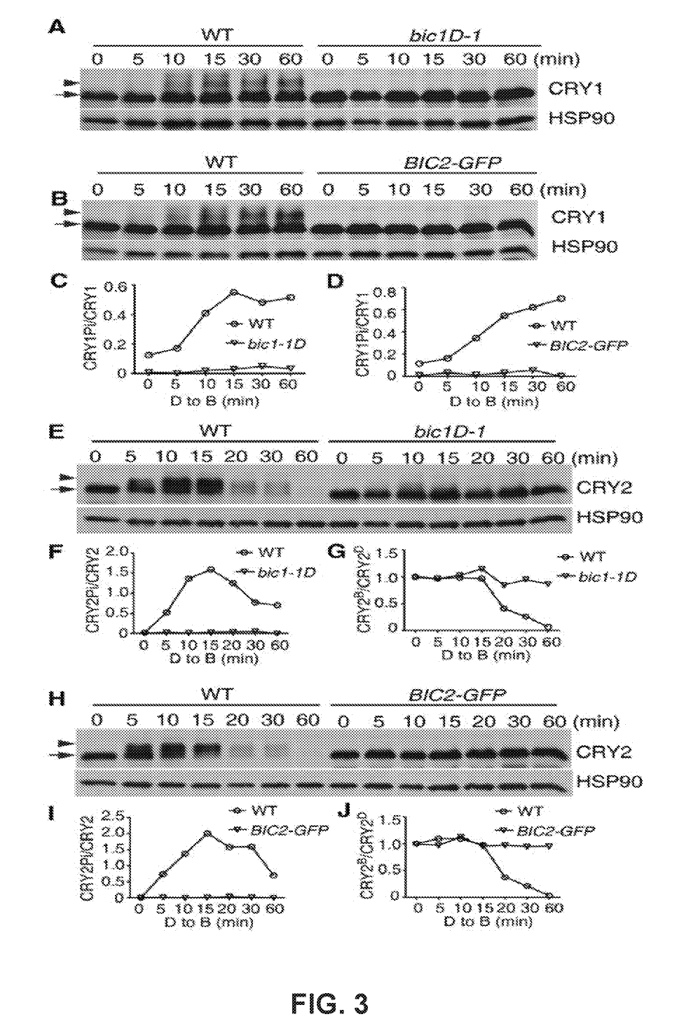

The cry 1 cry2 mutation and BIC1 overexpression caused similar transcriptome changes in response to blue light (FIG. 2 and table S2 of Wang et al), which suggests that BICs inhibit early photoreactions of CRYs. As reported previously (13-16), CRY1 and CRY2 underwent blue light-dependent phosphorylation and the phosphorylated CRY2 was degraded rapidly (FIG. 3, A to E, and FIG. S8 of Wang et al, upshifted bands). However, neither blue light-dependent phosphorylation of CRYs nor blue light-dependent degradation of CRY2 (15, 16) was detected in the plants overexpressing BIC1 or BIC2 (FIG. 3, A to E, and FIG. S8 of Wang et al); hence, BICs inhibit CRY phosphorylation.

Consistent with those results, the bic1bic2 mutant plants grown in blue or white light accumulated lower levels of CRY2 (FIG. 1, G to J of Wang et al), which seems physiologically hyperactive because the bic1bic2 mutant is hypersensitive to blue light (FIG. 1, A to C of Wang et al). The BIC-overexpressing plants grown in blue or white light accumulated higher levels of CRY2 (FIG. 1,G to J of Wang et al), which appears mostly inactive because the BIC-overexpressing plants are insensitive to blue light (FIG. 1, A to C of Wang et al).

BICs also inhibit the blue light-induced formation of CRY2 photobodies (FIG. 3 and FIG. S9 of Wang et al), which is another early photoreaction of CRY2 (17-19). FIG. 3 shows that CRY2-YFP (CRY2 fused to yellow fluorescent protein) formed photobodies within 60 s of blue-light exposure in the nucleus of the wild-type Arabidopsis protoplasts, whereas no CRY2-YFP photobodies were detected in the protoplasts overexpressing BIC1 or BIC2 after blue-light illumination for up to 60 min (FIGS. 3, F and H of Wang et al). In both darkness and light, BIC1 interacted with CRY2 in yeast or HEK293T (human embryonic kidney) cells via the conserved CRY interacting domain of BIC1 and the photolyase homologous region of CRY2 (FIGS. 4, B and F, and FIG. S10 of Wang et al). The results of the coimmunoprecipitation (co-IP) experiments indicate that blue light enhances BIC1-CRY2 interaction in plants. BIC1 coimmunoprecipitated CRY2 in seedlings exposed to blue light, but little BIC1-CRY2 complex was coprecipitated in the dark (FIG. 4A of Wang et al). This observation suggests that BIC1 might interact with photoexcited CRY2 to inhibit its activity.

Homodimers are the physiologically active form of plant CRYs (8, 9), but the effect of light on CRY dimerization has not been detected in previous studies (9, 20). This could be explained by, among other interpretations, light-independent CRY dimerization or masking effects of regulatory proteins, such as BICs, on the light-dependent CRY dimerization (9, 20). We reexamined the bluelight dependence of CRY2 dimerization using multiple approaches. In the first experiment, we coexpressed Flag-CRY2 and Myc-CRY2 in HEK293T cells (21-24) and tested the interaction between the two differentially tagged CRY2s by co-IP assay. In the absence of blue light, antibody to Flag coprecipitated little Myc-CRY2 from HEK293T cells expressing similar amounts of Flag-CRY2 and Myc-CRY2 (FIG. 4B and FIG. S12A of Wang et al).

In contrast, antibody to Flag coprecipitated increasing amounts of Myc-CRY2 from HEK293T cells exposed to blue light for 10 to 120 min, thereby demonstrating the light-dependent CRY2 homodimerization in the absence of BIC or other plant proteins (FIG. 4B and FIG. S12A of Wang et al). In a control experiment, human CRYs (hCRY1 and hCRY2) exhibited light-independent dimerization (FIG. 4C and FIG. S12D of Wang et al), which appears consistent with the light-independent activity of hCRYs in cultured HEK293T cells (25). The blue light-dependent CRY2 dimerization was also detected by the two hybrid assay in yeast cells (FIG. S11 of Wang et al) and the bimolecular fluorescence complementation (BiFC) assay in Arabidopsis protoplasts (FIGS. 3, G and I, and FIG. S9 of Wang et al). The BiFC assays revealed a more complex behavior of the intermolecular interaction of CRY2.

The BiFC signal resulting from the interaction between nYFP-CRY2 (N terminus of YFP fused to CRY2) and cCFP-CRY2 (C terminus of cyan fluorescent protein fused to CRY2) was detected regardless of blue-light treatment, whereas the fluorescent photobodies resulting from the interaction between nYFP-CRY2 and cCFP-CRY2 were detected only after blue-light treatment (FIGS. 3, G and I, and FIG. S9 of Wang et al).

Because photoexcited CRY2 is known to oligomerize into photobodies (18, 19, 23, 24), it is possible that in darkness nYFP-CRY2 and cCFPCRY2 interact weakly in a manner sufficient to reconstitute the fluorescent BiFC signal but insufficient to enable oligomerization of CRY2 into photobodies. In response to blue light, nYFPCRY2 and cCFP-CRY2 may interact with higher affinity to reconstitute not only the fluorescent BiFC signals but also fluorescent photobodies. To test this interpretation, we used co-IP assays to examine effects of blue light on CRY2 dimerization or oligomerization in plants coexpressing GFP-CRY2 (CRY2 fused to green fluorescent protein) and Myc-CRY2 (FIG. 4D of Wang et al). Antibody to GFP coprecipitated little Myc-CRY2 in etiolated seedlings, whereas the same antibody coprecipitated abundant Myc-CRY2 in etiolated seedlings exposed to blue light for 5 to 10 min (FIG. 4D of Wang et al). Similarly, the blue light-specific CRY2 homodimerization was also detected in adult plants (FIG. S12B of Wang et al). We conclude that the high-affinity CRY2 dimerization is a photoreaction in plant cells.

We next investigated the effects of BIC1 on blue light-dependent CRY2 dimerization using the multiple assays described above. We first examined dimerization of Flag-CRY2 and Myc-CRY2 in HEK293T cells coexpressing BIC1. FIG. 4B of Wang et al. shows that in the cells coexpressing BIC1, antibody to Flag coprecipitated only residual Myc-CRY2 even after blue-light treatment for up to 120 min, thereby demonstrating that BIC1 suppresses blue light-dependent CRY2 dimerization. The specificity of BIC1 inhibition of CRY2 dimerization is verified by the result that CIB1, which also interacts with photoexcited CRY2 (6, 26), did not inhibit blue light-dependent CRY2 dimerization (FIG. 4E of Wang et al.). The BIC1 inhibition of blue light-dependent CRY2 dimerization was also detected by the trihybrid assay in yeast cells (FIG. S11 of Wang et al.) and by the BiFC photobody assay in Arabidopsis cells (FIGS. 3, G and I, and FIG. S9 of Wang et al.). Because physiologically active CRY2 dimers or oligomers are expected to interact with their signaling partners, such as CIB1 and SPA1 (7), we used co-IP assays to test the effects of BIC1 on the blue light-dependent CRY2-CIB1 and CRY2-SPA1 interactions. As expected, coexpression of BIC1 suppressed the blue light-dependent CRY2-CIB1 interaction (FIG. 4F of Wang et al.) and CRY2-SPA1 interaction (FIG. S12C of Wang et al.), which explains how inhibition of CRY dimerization by BIC1 suppresses CRY2-dependent photoresponses of plants.

Homodimerization appears to be a common mechanism of photoreceptors (27-29). Our study provides evidence that plant CRYs exist as inactive monomers in the absence of light, whereas photoexcited CRYs form active homodimers or oligomers that interact with CRY-signaling proteins to trigger transcriptome changes responsible for photomorphogenesis; the BIC proteins interact with CRYs to prevent CRY homodimerization and thereby maintain the appropriate homeostasis of the active and inactive pools of CRYs and sustainability of cellular photosensitivity (FIG. S12E of Wang et al.). It would be interesting to examine whether photoinsensitive mammalian CRYs and photosensitive insect CRYs undergo circadian phase-dependent and light-dependent dimerization, respectively.

### EXAMPLE 1 REFERENCES

1. M. Ahmad, A. R. Cashmore, Nature 366, 162-166 (1993).
2. H. Guo, H. Yang, T. C. Mockler, C. Lin, Science 279, 1360-1363 (1998).
3. A. R. Cashmore, Cell 114, 537-543 (2003).
4. A. Sancar, Chem. Rev. 103, 2203-2237 (2003).
5. H.-Q. Yang et al., Cell 103, 815-827 (2000).
6. H. Liu et al., Science 322, 1535-1539 (2008).
7. H. Liu, B. Liu, C. Zhao, M. Pepper, C. Lin, Trends Plant Sci. 16, 684-691 (2011).
8. G. Rosenfeldt, R. M. Viana, H. D. Mootz, A. G. von Arnim, A. Batschauer, Mol. Plant 1, 4-14 (2008).
9. Y. Sang et al., Plant Cell 17, 1569-1584 (2005).
10. D. M. Shcherbakova, A. A. Shemetov, A. A. Kaberniuk, V. V. Verkhusha, Annu. Rev. Biochem. 84, 519-550 (2015).
11. T. Ichikawa et al., Plant J. 48, 974-985 (2006).
12. T. C. Mockler, H. Guo, H. Yang, H. Duong, C. Lin, Development 126, 2073-2082 (1999).
13. D. Shalitin et al., Nature 417, 763-767 (2002).
14. D. Shalitin, X. Yu, M. Maymon, T. Mockler, C. Lin, Plant Cell 15, 2421-2429 (2003).
15. Q. Wang et al., Mol. Plant 8, 631-643 (2015).
16. X. Yu et al., Plant Cell 19, 3146-3156 (2007).
17. P. Mas, P. F. Devlin, S. Panda, S. A. Kay, Nature 408, 207-211 (2000).
18. X. Yu et al., Plant Cell 21, 118-130 (2009).
19. L. J. Bugaj, A. T. Choksi, C. K. Mesuda, R. S. Kane, D. V. Schaffer, Nat. Methods 10, 249-252 (2013).
20. X. Yu et al., Proc. Natl. Acad. Sci. U.S.A. 104, 7289-7294 (2007).
21. J. Gao et al., Proc. Natl. Acad. Sci. U.S.A. 112, 9135-9140 (2015).
22. U. V. Pedmale et al., Cell 164, 233-245 (2016).
23. A. Taslimi et al., Nat. Commun. 5, 4925 (2014).
24. I. Ozkan-Dagliyan et al., J. Biol. Chem. 288, 23244-23251 (2013).
25. E. A. Griffin Jr., D. Staknis, C. J. Weitz, Science 286, 768-771 (1999).
26. M. J. Kennedy et al., Nat. Methods 7, 973-975 (2010).
27. L. Rizzini et al., Science 332, 103-106 (2011).
28. M. Heijde, R. Ulm, Proc. Natl. Acad. Sci. U.S.A. 110, 1113-1118 (2013).
29. C.-H. Chen, B. S. DeMay, A. S. Gladfelter, J. C. Dunlap, J. J. Loros, Proc. Natl. Acad. Sci. U.S.A. 107, 16715-16720 (2010).

### Example 2: Illustrative Method to Quantify Cry Homodimerization

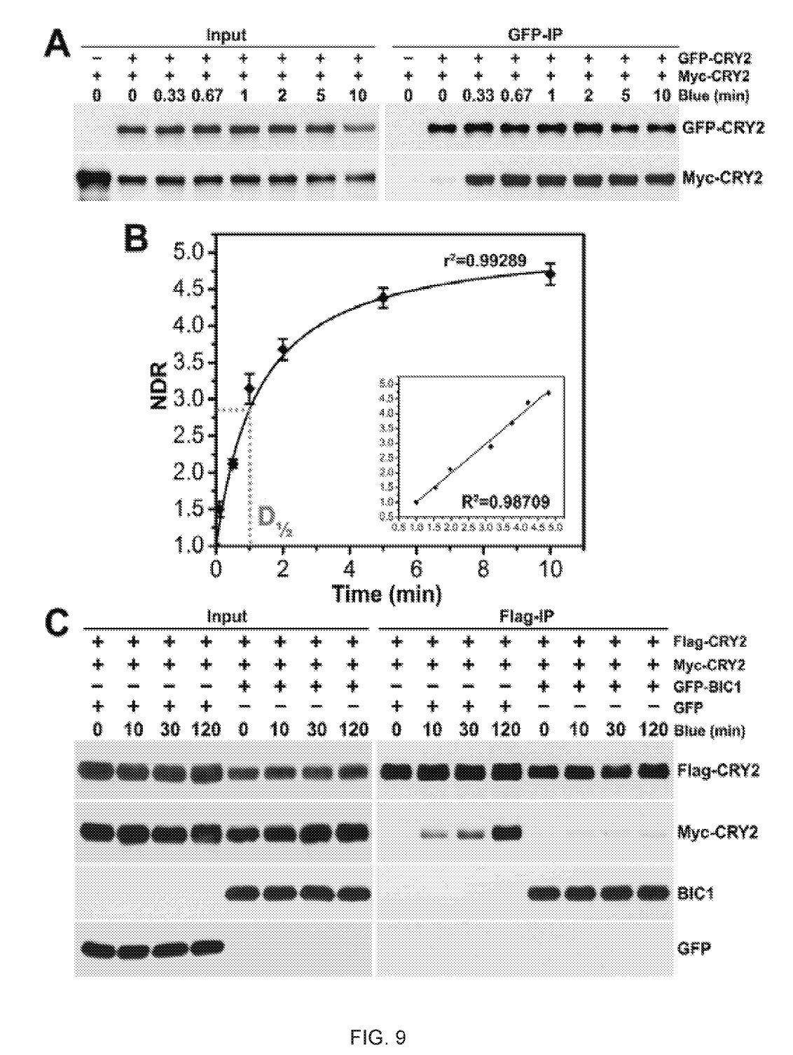

We designed a qCo-IP method to quantify CRY homodimerization (FIG. 9), and used this method to demonstrate that the photodimerization of Arabidopsis CRY2 occurs within 20 seconds upon light illumination. This result demonstrates an important utility of CRY2 photodimerization as a tool for optogenetics tools widely used in the study of human diseases and neurobiology.

As shown in FIG. 9, BIC1 inhibits blue-light dependent Arabidopsis CRY2 dimerization. Data in FIG. 9A shows blue light-dependent CRY2 dimerization in Arabidopsis. 7-day old etiolated seedlings coexpressing Myc-CRY2 and GFP-CRY2 were exposed to 30 μmol m⁻² s⁻¹ blue light for 20 sec (0.33 min), 40 sec (0.67 min), 1 min, 2 min, 5 min and 10 minutes. GFP-Trap-A were used to immunoprecipitate GFP-CRY2. GFP-CRY2 (IP signal) and Myc-CRY2 (co-IP signal) were detected by GFP or Myc antibody, respectively.

Data in FIG. 9B shows quantitative Co-IP analyses of CRY2 photodimerization in HEK293T cells. HEK293T cells co-expressing FLUC-CRY2 and REN-CRY2 were exposed to blue light (30 μmol·m⁻²·s⁻¹) for the time indicated, lysed, aliquots removed for the measurement of ATL (Adjusted Total Luminescence), and FLUC-CRY2 precipitated by anti-Flag antibody conjugated beads. Photodimerization of CRY2 was quantified by the dual-luciferase assay to calculate the Normalized Dimerization Ratio (NDR) by the formula: NDR=[(REN/LUC)L/(REN/LUC)D]/ATL, where LUC is the LUC luminescence detected for the precipitated FLUC-CRY2, REN is the REN luminescence detected for the co-precipitated REN-CRY2. (REN/LUC)L or (REN/LUC)D were measured in cells exposed to light or darkness, respectively. ATL (Adjusted Total Luminescence) is the cell volume-adjusted sum of LUC and REN luminescence of the cell lysates before immunoprecipitation, in which REN luminescence is converted to the LUC equivalent by the standard curve prepared by analyses of the LUC-REN fusion protein (not shown). ATL represents concentration of the CRY2 protein. Regression analysis of NDR as the function of time, SD (n=3), D1/2, and a correlation analysis (insert) of the results from two qCo-IP experiments under the same conditions are shown. The Maximum Photodimerization (Dm=4.7048) is defined by the maximum NDR; the Rate of Photodimerization (D1/2=1 min) is defined by the time needed to reach Dm.

Data in FIG. 9c shows HEK293T cells coexpressing Flag-CRY2, Myc-CRY2 and GFP-BIC1 or GFP were exposed to 180 μmol m⁻² s⁻¹ blue light for the time indicated. Anti-Flag antibody conjugated beads were used to perform the immunoprecipitations. Flag-CRY2 (IP signal) and Myc-CRY2 or GFP-BIC1 (co-IP signals) were detected by Flag and Myc or GFP antibody, respectively.

### Example 3: Cry Dimerization is Evolutionarily Conserved

Cryptochromes are the only photoreceptor that is evolutionarily conserved from bacteria to human, but the desensitization mechanism is first revealed by our discovery of BICs discussed herein this report. Cryptochrome is a critical component of the human circadian clock, which is associated with numerous human diseases, including diabetes, obesity, cancer, mania, etc. Our funding that BIC inhibits human CRY dimerization indicates that these plant proteins may be used to regulate the circadian clock in human, affecting potential treatment of various human diseases.

The light-dependent CRY2-CIB1 has been utilized as an optogenetics tool to achieve light-induced regulation of transcription, protein translocation, DNA recombination, phosphoinositide metabolism, epigenetics change, and reversible protein inactivation trap. Our discovery that BICs inhibits light-dependent CRY2-CIB1 interaction argues strongly that BIC can be used to control all those optogenetics reactions reported previously or in the future (FIG. 1B).

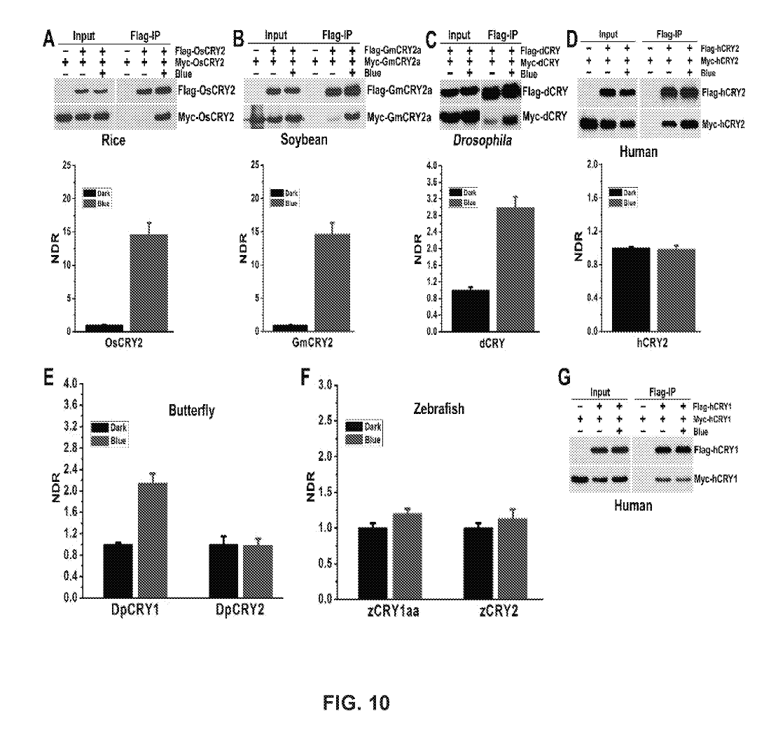

We show that, in addition to Arabidopsis CRY2, rice CRY2, soybean CRY2, Drosophila dCRY, and butterfly CRY1 also undergo photodimerization (light-dependent homodimerization), which are consistent with their published role as photoreceptors. In contract, human cryptochromes (hCRY1 and hCRY2), butterfly CRY2, and zebrafish CRYs undergo light-independent homodimerization, which are also consistent with their light-independent role in the circadian oscillator previously reported. These results demonstrate the evolutionary conservation and functional importance of homodimerization in evolutionary divergent organisms (see, e.g. FIG. 10). These results demonstrate the evolutionary conservation and functional importance of homodimerization in evolutionary divergent CRYs; and the ability of this system to be adapted for use in a wide range of biomedical applications.

Data in FIG. 10 shows dimerization activity of CRY from different organisms. Immuno blots: HEK293T cells coexpressing Flag-CRY and myc-CRY were exposed 25 to 100 μmol m⁻² s⁻¹ blue light for 2 hours (+) or kept in the dark (−). Anti-Flag antibody conjugated beads were used to perform the immunoprecipitations. Flag-CRY (IP signal) and Myc-CRY (co-IP signal) were detected by GFP or Myc antibody respectively.

For the data obtained in FIG. 10, qCo-IP: HEK293T cells co-expressing FLUC-CRY and REN-CRY were exposed to blue light (100 μmol·m⁻²·s⁻¹) for 2 hours. FLUC-CRY were precipitated by anti-Flag antibody conjugated beads. Photodimerization of CRY2 was quantified by the dual-luciferase assay to calculate the Normalized Dimerization Ratio (NDR) by the formula: NDR=[(REN/LUC)L/(REN/LUC)D]/ATL, where LUC is the LUC luminescence detected for the precipitated FLUC-CRY, REN is the REN luminescence detected for the co-precipitated REN-CRY. (REN/LUC)L or (REN/LUC)D were measured in cells exposed to light or darkness, respectively. ATL (Adjusted Total Luminescence) is the cell volume-adjusted sum of LUC and REN luminescence of the cell lysates before immunoprecipitation, in which REN luminescence is converted to the LUC equivalent by the standard curve prepared by analyses of the LUC-REN fusion protein.

### Example 4: Arabidopsis BIC1 Physically Interact with Human CRY1 and CRY2

Arabidopsis BIC1 physically interacts with human CRY1 and CRY2. The fact that plant BICs can physically interact with human cryptochromes argue for the potential utility of using plant BIC proteins to affect the activity of human CRYs and the circadian clock. Therefore, these results provide evidence for the utility of plant BICs as the molecular tools in the prevention and treatment of human diseases associated with human CRYs, such as cancer, diabetes, sleep disorder etc.

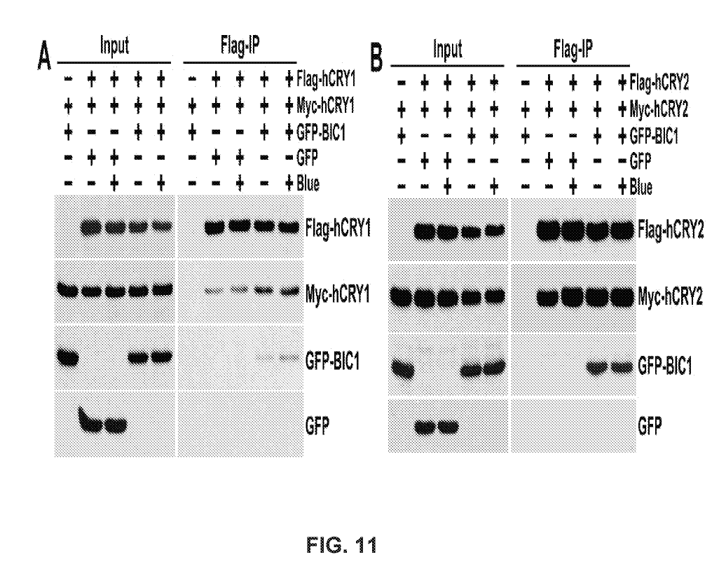

FIG. 11 provides data showing that Arabidopsis BIC1 interacts with both human CRY1 and CRY2, however the interactions have no effect on the dimerization activity of human CRYs.

In FIG. 11, HEK293T cells coexpressing Flag-hCRY, Myc-hCRY and GFP-BIC1 or GFP were exposed to 100 μmol m⁻² s⁻¹ blue light for 2 hours (+) or kept in the dark (−). Anti-Flag antibody conjugated beads were used to perform the immunoprecipitations. Flag-hCRY (IP signal) and Myc-hCRY or GFP-BIC1 (co-IP signals) were detected by Flag and Myc or GFP antibody, respectively. For the data obtained in FIG. 11, HEK293T cells coexpressing Flag-hCRY, Myc-hCRY and GFP-BIC1 or GFP were exposed to 100 μmol m⁻² s⁻¹ blue light for 2 hours (+) or kept in the dark (−). Anti-Flag antibody conjugated beads were used to perform the immunoprecipitations. Flag-hCRY (IP signal) and Myc-hCRY or GFP-BIC1 (co-IP signals) were detected by Flag and Myc or GFP antibody, respectively.

SEQUENCE LISTINGS
BIC1 DNA SEQUENCE AT3G52740 (SEQ ID NO: 1)

| | |
|---|---|
| 1 | TCAACACCGA ATCTCTCAAC ACAAACAAAA TCACACATCT |
| | CTCTTCATCT |
| 51 | TTTTGTTTCC TGCAAGAATC TGAATCTGCT TTACTATTGT |
| | GTCATCATGA |
| 101 | TGAACATCGA CGATACGACG TCTCCAATGG CCCACCCGAT |
| | CGGTCCATCT |
| 151 | CAGCCTCCTT CCGACCAAAC CAAACAAGAT CCGCCAAGTT |
| | TGCCCCAAGA |
| 201 | AGCAGCTTCT TCTGTTTCGG CCGACAAGAA AGATCTAGCT |
| | TTGCTTGAAG |
| 251 | AGAAACCGAA GCAGAGTCAA GAAGAAGATA GAGTGGACAC |
| | TGGGAGAGAG |
| 301 | AGGTTAAAGA AGCATCGGAG AGAGATCGCT GGTAGGGTTT |
| | GGATACCGGA |
| 351 | GATATGGGGA CAAGAAGAGC TTCTTAAGGA TTGGATCGAT |
| | TGTTCAACGT |
| 401 | TTGACACGTG TCTAGTCCCT GCCGGAATCT CGTCTGCACG |
| | TACTGCTCTC |
| 451 | GTAGAGGAAG CTAGGCGAGC TGCTTCAGCT TCTGGTGGGT |
| | TACATAATCG |
| 501 | TTGCTTGATC TTACGTTGAA TTTAATATAA TAAGATAACA |
| | TACTTATAAA |
| 551 | TGTGGTTCTT GTTCCTCAAT AATATAAGGC ACTATTGTTA |
| | C |

BIC1 PROTEIN SEQUENCE (SEQ ID NO: 2)

| | |
|---|---|
| 1 | MMNIDDTTSP MAHPIGPSQP PSDQTKQDPP SLPQEAASSV |
| | SADKKDLALL |
| 51 | EEKPKQSQEE DRVDTGRERL KKHRREIAGR VWIPEIWGQE |
| | ELLKDWIDCS |
| 101 | TFDTCLVPAG ISSARTALVE EARRAASASG GLHNRCLILR |

BIC2 DNA SEQUENCE AT3G44450 (SEQ ID NO: 3)

| | |
|---|---|
| 1 | TATTTCTCTC TAAATCTCAA CATTTTTATA TATACAACAC |
| | ACACGTCGAA |
| 51 | GCCAATAAAA ATCTCTACTA CAAAAGTAAA AATAAAGAAA |
| | AGAGTTCAAA |
| 101 | ATGAAGAACA CCAATTTGCC TGAAGAAACC AAGGAACCAA |
| | TCTCTCCAGG |
| 151 | ATCTTCTCAC CGGAAACAAA ACAAGACAGG TACAAAGACA |
| | TGTTTCCCGG |
| 201 | AAACAACGGT GTTGTCAGGA CGTGATAGGC TAAAGAGACA |
| | TAGAGAAGAG |
| 251 | GTTGCCGGAA AAGTTCCTAT ACCGGATAGT TGGGGAAAAG |
| | AAGGATTGCT |
| 301 | TATGGGATGG ATGGATTTTT CGACCTTCGA CGCTGCTTTT |
| | ACGTCTAGCC |
| 351 | AGATTGTCTC TGCTCGAGCT GCGTTAATGG CTGACTCAGG |
| | AGACGATGCC |
| 401 | GGAGCTAGAG GAAGTAGGCC TCAACGCCTT CGAGTTGAGA |
| | GTTCTTGTTG |
| 451 | ATTTCATTGT CTTAGAGAAA TGTTTATGAA ATAATACTCT |
| | CATCATGATT |
| 501 | TTGAATGTTA TTATTAGATA CCTTATCTTC ACATTGATTT |
| | TGAAATTTTG |
| 551 | AACCATTGAT ACAATTATTC AAAGTTTCAT ATA |

BIC2 PROTEIN SEQUENCE (SEQ ID NO: 4)

| | |
|---|---|
| 1 | MKNTNLPEET KEPISPGSSH RKQNKTGTKT CFPETTVLSG |
| | RDRLKRHREE |
| 51 | VAGKVPIPDS WGKEGLLMGW MDFSTFDAAF TSSQIVSARA |
| | ALMADSGDDA |
| 101 | GARGSRPQRL RVESSC |

CRY2 DNA SEQUENCE AT1G04400 (SEQ ID NO: 5)

| | |
|---|---|
| 1 | ATGAAGATGG ACAAAAAGAC TATAGTTTGG TTTAGAAGAG |
| | ACCTAAGGAT |
| 51 | TGAGGATAAT CCTGCATTAG CAGCAGCTGC TCACGAAGGA |
| | TCTGTTTT TC |
| 101 | CTGTCTTCAT TTGGTGTCCT GAAGAAGAAG GACAGTTTTA |
| | TCCTGGAA GA |
| 151 | GCTTCAAGAT GGTGGATGAA ACAATCACTT GCTCACTTAT |
| | CTCAATCC TT |
| 201 | GAAGGCTCTT GGATCTGACC TCACTTTAAT CAAAACCCAC |
| | AACACGAT TT |
| 251 | CAGCGATCTT GGATTGTATC CGCGTTACCG GTGCTACAAA |
| | AGTCGTCT TT |
| 301 | AACCACCTCT ATGATCCTGT TTCGTTAGTT CGGGACCATA |
| | CCGTAAAG GA |
| 351 | GAAGCTGGTG GAACGTGGGA TCTCTGTGCA AAGCTACAAT |
| | GGAGATCT AT |
| 401 | TGTATGAACC GTGGGAGATA TACTGCGAAA AGGGCAAACC |
| | TTTTACGA GT |
| 451 | TTCAATTCTT ACTGGAAGAA ATGCTTAGAT ATGTCGATTG |
| | AATCCGTT AT |
| 501 | GCTTCCTCCT CCTTGGCGGT TGATGCCAAT AACTGCAGCG |
| | GCTGAAGC GA |
| 551 | TTTGGGCGTG TTCGATTGAA GAACTAGGGC TGGAGAATGA |
| | GGCCGAGA AA |
| 601 | CCGAGCAATG CGTTGTTAAC TAGAGCTTGG TCTCCAGGAT |
| | GGAGCAAT GC |
| 651 | TGATAAGTTA CTAAATGAGT TCATCGAGAA GCAGTTGATA |
| | GATTATGC AA |
| 701 | AGAACAGCAA GAAAGTTGTT GGGAATTCTA CTTCACTACT |
| | TTCTCCGT AT |
| 751 | CTCCATTTCG GGGAAATAAG CGTCAGACAC GTTTTCCAGT |
| | GTGCCCGG AT |
| 801 | GAAACAAATT ATATGGGCAA GAGATAAGAA CAGTGAAGGA |
| | GAAGAAAG TG |
| 851 | CAGATCTTTT TCTTAGGGGA ATCGGTTTAA GAGAGTATTC |
| | TCGGTATA TA |
| 901 | TGTTTCAACT TCCCGTTTAC TCACGAGCAA TCGTTGTTGA |
| | GTCATCTT CG |
| 951 | GTTTTTCCCT TGGGATGCTG ATGTTGATAA GTTCAAGGCC |
| | TGGAGACA AG |
| 1001 | GCAGGACCGG TTATCCGTTG GTGGATGCCG GAATGAGAGA |
| | GCTTTGGG CT |
| 1051 | ACCGGATGGA TGCATAACAG AATAAGAGTG ATTGTTTCAA |
| | GCTTTGCT GT |
| 1101 | GAAGTTTCTT CTCCTTCCAT GGAAATGGGG AATGAAGTAT |
| | TTCTGGGA TA |
| 1151 | CACTTTTGGA TGCTGATTTG GAATGTGACA TCCTTGGCTG |
| | GCAGTATA TC |
| 1201 | TCTGGGAGTA TCCCCGATGG CCACGAGCTT GATCGCTTGG |
| | ACAATCCC GC |
| 1251 | GTTACAAGGC GCCAAATATG ACCCAGAAGG TGAGTACATA |
| | AGGCAATG GC |
| 1301 | TTCCCGAGCT TGCGAGATTG CCAACTGAAT GGATCCATCA |
| | TCCATGGG AC |
| 1351 | GCTCCTTTAA CCGTACTCAA AGCTTCTGGT GTGGAACTCG |
| | GAACAAAC TA |
| 1401 | TGCGAAACCC ATTGTAGACA TCGACACAGC TCGTGAGCTA |
| | CTAGCTAA AG |
| 1451 | CTATTTCAAG AACCCGTGAA GCACAGATCA TGATCGGAGC |
| | AGCACCTG AT |
| 1501 | GAGATTGTAG CAGATAGCTT CGAGGCCTTA GGGGCTAATA |
| | CCATTAAA GA |
| 1551 | ACCTGGTCTT TGCCCATCTG TGTCTTCTAA TGACCAACAA |
| | GTACCTTC GG |
| 1601 | CTGTTCGTTA CAACGGGTCA AAGAGAGTGA AACCTGAGGA |
| | AGAAGAAG AG |
| 1651 | AGAGACATGA AGAAATCTAG GGGATTCGAT GAAAGGGAGT |
| | TGTTTTCG AC |
| 1701 | TGCTGAATCT TCTTCTTCTT CGAGTGTGTT TTTCGTTTCG |
| | CAGTCTTG CT |
| 1751 | CGTTGGCATC AGAAGGGAAG AATCTGGAAG GTATTCAAGA |
| | TTCATCTG AT |
| 1801 | CAGATTACTA CAAGTTTGGG AAAAAATGGT TGCAAATGA |

CRY2 PROTEIN SEQUENCE (SEQ ID NO: 6)

| | |
|---|---|
| 1 | MKMDKKTIVW FRRDLRIEDN PALAAAAHEG SVFPVFIWCP |
| | EEEGQFYPGR |
| 51 | ASRWWMKQSL AHLSQSLKAL GSDLTLIKTH NTISAILDCI |
| | RVTGATKVV F |
| 101 | NHLYDPVSLV RDHTVKEKLV ERGISVQSYN GDLLYEPWEI |
| | YCEKGKPFT S |
| 151 | FNSYWKKCLD MSIESVMLPP PWRLMPITAA AEAIWACSIE |
| | ELGLENEAE K |
| 201 | PSNALLTRAW SPGWSNADKL LNEFIEKQLI DYAKNSKKVV |
| | GNSTSLLSP Y |
| 251 | LHFGEISVRH VFQCARMKQI IWARDKNSEG EESADLFLRG |
| | IGLREYSRY I |
| 301 | CFNFPFTHEQ SLLSHLRFFP WDADVDKFKA WRQGRTGYPL |
| | VDAGMRELW A |
| 351 | TGWMHNRIRV IVSSFAVKFL LLPWKWGMKY FWDTLLDADL |
| | ECDILGWQY I |
| 401 | SGSIPDGHEL DRLDNPALQG AKYDPEGEYI RQWLPELARL |
| | PTEWIHHPW D |
| 451 | APLTVLKASG VELGTNYAKP IVDIDTAREL LAKAISRTRE |
| | AQIMIGAAP D |
| 501 | EIVADSFEAL GANTIKEPGL CPSVSSNDQQ VPSAVRYNGS |
| | KRVKPEEEE E |
| 551 | RDMKKSRGFD ERELFSTAES SSSSSVFFVS QSCSLASEGK |
| | NLEGIQDSS D |
| 601 | QITTSLGKNG CK |

CIB1 DNA SEQUENCE AT4G34530 (SEQ ID NO: 7)

| | |
|---|---|
| 1 | ATGAATGGAG CTATAGGAGG TGACCTTTTG CTCAATTTTC |
| | CTGACATGTC |
| 51 | GGTCCTAGAG CGCCAAAGGG CTCACCTCAA GTACCTCAAT |
| | CCCACCTT TG |
| 101 | ATTCTCCTCT CGCCGGCTTC TTTGCCGATT CTTCAATGAT |
| | TACCGGCG GC |
| 151 | GAGATGGACA GCTATCTTTC GACTGCCGGT TTGAATCTTC |
| | CGATGATG TA |
| 201 | CGGTGAGACG ACGGTGGAAG GTGATTCAAG ACTCTCAATT |
| | TCGCCGGA AA |
| 251 | CGACGCTTGG GACTGGAAAT TTCAAGAAAC GGAAGTTTGA |
| | TACAGAGA CT |
| 301 | AAGGATTGTA ATGAGAAGAA GAAGAAGATG ACGATGAACA |
| | GAGATGAC CT |
| 351 | AGTAGAAGAA GGAGAAGAAG AGAAGTCGAA AATAACAGAG |
| | CAAAACAA TG |
| 401 | GGAGCACAAA AAGCATCAAG AAGATGAAAC ACAAAGCCAA |
| | GAAAGAAG AG |
| 451 | AACAATTTCT CTAATGATTC ATCTAAAGTG ACGAAGGAAT |
| | TGGAGAAA AC |
| 501 | GGATTATATT CATGTTCGTG CACGACGAGG CCAAGCCACT |
| | GATAGTCA CA |
| 551 | GCATAGCAGA ACGAGTTAGA AGAGAAAAGA TCAGTGAGAG |
| | AATGAAGT TT |
| 601 | CTACAAGATT TGGTTCCTGG ATGCGACAAG ATCACAGGCA |
| | AAGCAGGG AT |
| 651 | GCTTGATGAA ATCATTAACT ATGTTCAGTC TCTTCAGAGA |
| | CAAATCGA GT |
| 701 | TCTTATCGAT GAAACTAGCA ATTGTGAATC CAAGGCCGGA |
| | TTTTGATA TG |
| 751 | GATGACATTT TTGCCAAAGA GGTTGCCTCA ACTCCAATGA |
| | CTGTGGTG CC |
| 801 | ATCTCCTGAA ATGGTTCTTT CCGGTTATTC TCATGAGATG |
| | GTTCACTC TG |
| 851 | GTTATTCTAG TGAGATGGTT AACTCCGGTT ACCTTCATGT |
| | CAATCCAA TG |
| 901 | CAGCAAGTGA ATACCAGTTC TGATCCATTG TCATGCTTCA |
| | ACAATGGC GA |
| 951 | AGCTCCTTCG ATGTGGGACT CTCATGTGCA GAATCTCTAT |
| | GGCAATTT AG |
| 1001 | GAGTTTGA |

CIB1 PROTEIN SEQUENCE (SEQ ID NO: 8)

| | |
|---|---|
| 1 | MNGAIGGDLL LNFPDMSVLE RQRAHLKYLN PTFDSPLAGF |
| | FADSSMITGG |
| 51 | EMDSYLSTAG LNLPMMYGET TVEGDSRLSI SPETTLGTGN |
| | FKKRKFDTE T |
| 101 | KDCNEKKKKM TMNRDDLVEE GEEEKSKITE QNNGSTKSIK |
| | KMKHKAKKE E |
| 151 | NNFSNDSSKV TKELEKTDYI HVRARRGQAT DSHSIAERVR |
| | REKISERMK F |
| 201 | LQDLVPGCDK ITGKAGMLDE IINYVQSLQR QIEFLSMKLA |
| | IVNPRPDFD M |
| 251 | DDIFAKEVAS TPMTVVPSPE MVLSGYSHEM VHSGYSSEMV |
| | NSGYLHVNP M |
| 301 | QQVNTSSDPL SCFNNGEAPS MWDSHVQNLY GNLGV |

### REFERENCES

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.
1. Liu, Hongtao, et al. (2008). Photoexcited CRY2 interacts with CIB1 to regulate transcription and floral initiation in Arabidopsis. Science 322, 1535-1539.
2. Shimizu-Sato, S., Huq, E., Tepperman, J. M., and Quail, P. H. (2002). A light-switchable gene promoter system. Nat Biotechnol 20, 1041-1044.
3. Davidson, E. A., Scouras, A., Ellington, A. D., Marcotte, E. M., and Voigt, C. A. (2005). Synthetic biology: engineering Escherichia coli to see light. Nature 438, 441-442.

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A composition of matter comprising:
a polynucleotide encoding a blue-light inhibitor of cryptochrome (BIC) polypeptide; wherein:
the polynucleotide encoding the BIC polypeptide is coupled to heterologous nucleic acids comprising a promoter; and
the BIC polypeptide inhibits the light dependent function of a cryptochrome polypeptide.

2. The composition of claim 1, wherein the BIC polypeptide has an at least 90% amino acid sequence identity to SEQ ID NO: 2 or SEQ ID NO: 4.

3. The composition of claim 1, wherein the BIC polypeptide inhibits blue-light dependent dimerization of cryptochrome 2 polypeptide (SEQ ID NO: 6).

4. The composition of claim 1, wherein the BIC polypeptide is coupled to a heterologous amino acid segment.

5. The composition of claim 1, wherein the polynucleotide encoding the BIC polypeptide is a transgene that expresses the BIC polypeptide within a cell.

6. The composition of claim 5, wherein the cell is a plant cell or a mammalian cell.

7. The composition of claim 1, wherein the composition further comprises a polynucleotide encoding a cryptochrome polypeptide.

8. The composition of claim 7, wherein the cryptochrome polypeptide has an at least 90% amino acid sequence identity to SEQ ID NO: 6.

9. The composition of claim 1, wherein the promoter is an inducible promoter.

10. A composition of matter comprising:
(a) a cryptochrome (CRY) polypeptide;
(b) a cryptochrome polypeptide-interacting basic helix-loop-helix (CIB) polypeptide; and
(c) a blue-light inhibitor of cryptochrome (BIC) polypeptide; wherein:
at least one of the polypeptides of (a)-(c) is coupled to a heterologous amino acid segment.

11. The composition of claim 10, wherein:
the BIC polypeptide has an at least 90% amino acid sequence identity to SEQ ID NO: 2 or SEQ ID NO: 4;
the CIB polypeptide has an at least 90% amino acid sequence identity to SEQ ID NO: 8; and/or
the CRY polypeptide has an at least 90% amino acid sequence identity to SEQ ID NO: 6.

12. The composition of claim 11, further comprising Flavin adenine dinucleotide (FAD).

13. The composition of claim 10, wherein the composition is disposed in an in vitro environment.

14. The composition of claim 10, wherein the composition is disposed in a mammalian cell.

15-16. (canceled)

17. An optogenetic system comprising:
a vessel comprising one or more compartments containing:
a cryptochrome (CRY) protein;
a cryptochrome polypeptide-interacting basic helix-loop-helix (CIB) protein; and
a blue-light inhibitor of cryptochrome (BIC) protein;
an aqueous solution disposed within the vessel; and
a blue light source.

18. The system of claim 17, wherein:
the CRY polypeptide has an at least 90% amino acid sequence identity to SEQ ID NO: 6;
the CIB polypeptide has an at least 90% amino acid sequence identity to SEQ ID NO: 8; and/or
the BIC polypeptide has an at least 90% amino acid sequence identity to SEQ ID NO: 2 or SEQ ID NO: 4.

19. The system of claim 17, wherein the system further comprises a cell culture media.

20. The system of claim 17. wherein the system further comprises mammalian cells.


SEQUENCE LISTING (8 sequences)

SEQ ID NO: 1 - DNA, 591 bp, Arabidopsis thaliana
tcaacaccgaatctctcaacacaaacaaaatcacacatctctcttcatctttttgtttcctgcaagaatctgaatctgctttactattgtgtcatcatgatgaacatcgacgatacgacgtctccaatggcccacccgatcggtccatctcagcctccttccgaccaaaccaaacaagatccgccaagtttgccccaagaagcagcttcttctgtttcggccgacaagaaagatctagctttgcttgaagagaaaccgaagcagagtcaagaagaagatagagtggacactgggagagagaggttaaagaagcatcggagagagatcgctggtagggtttggataccggagatatggggacaagaagagcttcttaaggattggatcgattgttcaacgtttgacacgtgtctagtccctgccggaatctcgtctgcacgtactgctctcgtagaggaagctaggcgagctgcttcagcttctggtgggttacataatcgttgcttgatcttacgttgaatttaatataataagataacatacttataaatgtggttcttgttcctcaataatataaggcactattgttac

SEQ ID NO: 2 - protein, 140 aa, Arabidopsis thaliana
MMNIDDTTSPMAHPIGPSQPPSDQTKQDPPSLPQEAASSVSADKKDLALLEEKPKQSQEEDRVDTGRERLKKHRREIAGRVWIPEIWGQEELLKDWIDCSTFDTCLVPAGISSARTALVEEARRAASASGGLHNRCLILR

SEQ ID NO: 3 - DNA, 583 bp, Arabidopsis thaliana
tatttctctctaaatctcaacatttttatatatacaacacacacgtcgaagccaataaaaatctctactacaaaagtaaaaataaagaaaagagttcaaaatgaagaacaccaatttgcctgaagaaaccaaggaaccaatctctccaggatcttctcaccggaaacaaaacaagacaggtacaaagacatgtttcccggaaacaacggtgttgtcaggacgtgataggctaaagagacatagagaagaggttgccggaaaagttcctataccggatagttggggaaaagaaggattgcttatgggatggatggatttttcgaccttcgacgctgcttttacgtctagccagattgtctctgctcgagctgcgttaatggctgactcaggagacgatgccggagctagaggaagtaggcctcaacgccttcgagttgagagttcttgttgatttcattgtcttagagaaatgtttatgaaataatactctcatcatgattttgaatgttattattagataccttatcttcacattgattttgaaattttgaaccattgatacaattattcaaagtttcatata

SEQ ID NO: 4 - protein, 116 aa, Arabidopsis thaliana
MKNTNLPEETKEPISPGSSHRKQNKTGTKTCFPETTVLSGRDRLKRHREEVAGKVPIPDSWGKEGLLMGWMDFSTFDAAFTSSQIVSARAALMADSGDDAGARGSRPQRLRVESSC

SEQ ID NO: 5 - DNA, 1839 bp, Arabidopsis thaliana
atgaagatggacaaaaagactatagtttggtttagaagagacctaaggattgaggataatcctgcattagcagcagctgctcacgaaggatctgtttttcctgtcttcatttggtgtcctgaagaagaaggacagttttatcctggaagagcttcaagatggtggatgaaacaatcacttgctcacttatctcaatccttgaaggctcttggatctgacctcactttaatcaaaacccacaacacgatttcagcgatcttggattgtatccgcgttaccggtgctacaaaagtcgtctttaaccacctctatgatcctgtttcgttagttcgggaccataccgtaaaggagaagctggtggaacgtgggatctctgtgcaaagctacaatggagatctattgtatgaaccgtgggagatatactgcgaaaagggcaaaccttttacgagtttcaattcttactggaagaaatgcttagatatgtcgattgaatccgttatgcttcctcctccttggcggttgatgccaataactgcagcggctgaagcgatttgggcgtgttcgattgaagaactagggctggagaatgaggccgagaaaccgagcaatgcgttgttaactagagcttggtctccaggatggagcaatgctgataagttactaaatgagttcatcgagaagcagttgatagattatgcaaagaacagcaagaaagttgttgggaattctacttcactactttctccgtatctccatttcggggaaataagcgtcagacacgttttccagtgtgcccggatgaaacaaattatatgggcaagagataagaacagtgaaggagaagaaagtgcagatctttttcttaggggaatcggtttaagagagtattctcggtatatatgtttcaacttcccgtttactcacgagcaatcgttgttgagtcatcttcggtttttcccttgggatgctgatgttgataagttcaaggcctggagacaaggcaggaccggttatccgttggtggatgccggaatgagagagctttgggctaccggatggatgcataacagaataagagtgattgtttcaagctttgctgtgaagtttcttctccttccatggaaatggggaatgaagtatttctgggatacacttttggatgctgatttggaatgtgacatccttggctggcagtatatctctgggagtatccccgatggccacgagcttgatcgcttggacaatcccgcgttacaaggcgccaaatatgacccagaaggtgagtacataaggcaatggcttcccgagcttgcgagattgccaactgaatggatccatcatccatgggacgctcctttaaccgtactcaaagcttctggtgtggaactcggaacaaactatgcgaaacccattgtagacatcgacacagctcgtgagctactagctaaagctatttcaagaacccgtgaagcacagatcatgatcggagcagcacctgatgagattgtagcagatagcttcgaggccttaggggctaataccattaaagaacctggtctttgcccatctgtgtcttctaatgaccaacaagtaccttcggctgttcgttacaacgggtcaaagagagtgaaacctgaggaagaagaagagagagacatgaagaaatctaggggattcgatgaaagggagttgttttcgactgctgaatcttcttcttcttcgagtgtgtttttcgtttcgcagtcttgctcgttggcatcagaagggaagaatctggaaggtattcaagattcatctgatcagattactacaagtttgggaaaaaatggttgcaaatga

SEQ ID NO: 6 - protein, 612 aa, Arabidopsis thaliana
MKMDKKTIVWFRRDLRIEDNPALAAAAHEGSVFPVFIWCPEEEGQFYPGRASRWWMKQSLAHLSQSLKALGSDLTLIKTHNTISAILDCIRVTGATKVVFNHLYDPVSLVRDHTVKEKLVERGISVQSYNGDLLYEPWEIYCEKGKPFTSFNSYWKKCLDMSIESVMLPPPWRLMPITAAAEAIWACSIEELGLENEAEKPSNALLTRAWSPGWSNADKLLNEFIEKQLIDYAKNSKKVVGNSTSLLSPYLHFGEISVRHVFQCARMKQIIWARDKNSEGEESADLFLRGIGLREYSRYICFNFPFTHEQSLLSHLRFFPWDADVDKFKAWRQGRTGYPLVDAGMRELWATGWMHNRIRVIVSSFAVKFLLLPWKWGMKYFWDTLLDADLECDILGWQYISGSIPDGHELDRLDNPALQGAKYDPEGEYIRQWLPELARLPTEWIHHPWDAPLTVLKASGVELGTNYAKPIVDIDTARELLAKAISRTREAQIMIGAAPDEIVADSFEALGANTIKEPGLCPSVSSNDQQVPSAVRYNGSKRVKPEEEEERDMKKSRGFDERELFSTAESSSSSSVFFVSQSCSLASEGKNLEGIQDSSDQITTSLGKNGCK

SEQ ID NO: 7 - DNA, 1008 bp, Arabidopsis thaliana
atgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaagaaacggaagtttgatacagagactaaggattgtaatgagaagaagaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcatgttcgtgcacgacgaggccaagccactgatagtcacagcatagcagaacgagttagaagagaaaagatcagtgagagaatgaagtttctacaagatttggttcctggatgcgacaagatcacaggcaaagcagggatgcttgatgaaatcattaactatgttcagtctcttcagagacaaatcgagttcttatcgatgaaactagcaattgtgaatccaaggccggattttgatatggatgacatttttgccaaagaggttgcctcaactccaatgactgtggtgccatctcctgaaatggttctttccggttattctcatgagatggttcactctggttattctagtgagatggttaactccggttaccttcatgtcaatccaatgcagcaagtgaataccagttctgatccattgtcatgcttcaacaatggcgaagctccttcgatgtgggactctcatgtgcagaatctctatggcaatttaggagtttga

SEQ ID NO: 8 - protein, 335 aa, Arabidopsis thaliana
MNGAIGGDLLLNFPDMSVLERQRAHLKYLNPTFDSPLAGFFADSSMITGGEMDSYLSTAGLNLPMMYGETTVEGDSRLSISPETTLGTGNFKKRKFDTETKDCNEKKKKMTMNRDDLVEEGEEEKSKITEQNNGSTKSIKKMKHKAKKEENNFSNDSSKVTKELEKTDYIHVRARRGQATDSHSIAERVRREKISERMKFLQDLVPGCDKITGKAGMLDEIINYVQSLQRQIEFLSMKLAIVNPRPDFDMDDIFAKEVASTPMTVVPSPEMVLSGYSHEMVHSGYSSEMVNSGYLHVNPMQQVNTSSDPLSCFNNGEAPSMWDSHVQNLYGNLGV